(19) United States
(12) Patent Application Publication
(10) Pub. No.: US 2005/0054044 A1
(43) Pub. Date: Mar. 10, 2005

(54) Method of alleviating nucleotide limitations for in vitro protein synthesis

(75) Inventors: James Robert Swartz, Stanford, CA (US); Michael Christopher Jewett, Stanford, CA (US); Kara Anne Calhoun, Stanford, CA (US)

Correspondence Address: BOZICEVIC, FIELD & FRANCIS LLP, 1900 UNIVERSITY AVE, SUITE 200, EAST PALO ALTO, CA, 94303, US

(73) Assignee: The Board of Trustees of the Leland Stanford Junior University

(21) Appl. No.: 10/888,303

(22) Filed: Jul. 9, 2004

Related U.S. Application Data
(60) Provisional application No. 60/488,264, filed on Jul. 18, 2003.

(51) Int. Cl. C12P 21/02

(52) U.S. Cl. 435/69.1; 435/320.1; 435/325; 530/352

(57) ABSTRACT

Compositions and methods are provided for the improved in vitro synthesis of polypeptides, where the duration of detectable protein synthesis in a reaction is substantially extended over existing methods, thereby providing for increased total yield of polypeptide. Increased synthesis is accomplished by maintaining the concentration of CTP and UTP in the reaction mixture at a pre-determined level. In another embodiment of the invention, increased synthesis is obtained by maintaining the concentration of cysteine and serine at a pre-determined level. The reaction mixture may be supplemented with additional amino acids during the course of the reaction.

### BACKGROUND OF THE INVENTION

Protein synthesis is a fundamental biological process, which underlies the development of polypeptide therapeutics, diagnostics, and catalysts. With the advent of recombinant DNA (rDNA) technology, it has become possible to harness the catalytic machinery of the cell to produce a desired protein. This can be achieved within the cellular environment or in vitro using extracts derived from cells.

There is a growing need for efficient protein production technologies. Cell-free protein synthesis offers an attractive and convenient approach to produce properly folded recombinant DNA (rDNA) proteins on a laboratory scale, incorporate unnatural or labeled amino acids into proteins, screen PCR fragment libraries in a high-throughput format, and express pharmaceutical proteins. The well controlled and flexible environment offers several advantages over conventional in vivo technologies.

Cell-free systems can direct most, if not all, of the available metabolic resources towards the exclusive production of one protein. Moreover, the lack of a cell wall and membrane components in vitro is advantageous since it allows for control of the synthesis environment. For example, tRNA levels can be changed to reflect the codon usage of genes being expressed. The redox potential, pH, or ionic strength can also be altered with greater flexibility than in vivo since we are not concerned about cell growth or viability. Furthermore, direct recovery of purified, properly folded protein products can be easily achieved.

In vitro translation is also recognized for its ability to incorporate unnatural and isotope-labeled amino acids as well as its capability to produce proteins that are unstable, insoluble, or cytotoxic in vivo. In addition, cell-free protein synthesis may play a role in revolutionizing protein engineering and proteomic screening technologies. The cell-free method bypasses the laborious processes required for cloning and transforming cells for the expression of new gene products in vivo and is becoming a platform technology for this field.

Although there have been tremendous efforts in making in vitro biosynthesis an appealing method for protein expression, this approach is still limited by short reaction times and low protein production rates. For example, the protein production rate of in vitro translation systems has improved two-orders of magnitude over the past ten years to about 500 μg protein/ml-hr. Although significant, these efforts fall short of effectively capturing nature's astounding potential. The internal protein production rate of a rapidly growing bacterium can reach up to 400 mg total Escherichia coli protein/ml-hr, assuming a 20-minute doubling time and 200 mg/ml cytoplasmic protein concentration. Cell-free recombinant DNA protein synthesis rates are at least 1000-fold below this production capability.

In addition to protein production rates, the duration for protein synthesis systems has also improved dramatically. Batch reactions have increased from a mere twenty minutes with the conventional PEP system to up to six hours. However the termination of protein synthesis after six hours still limits this technology.

Increasing the production rates and/or the duration of protein biosynthesis would be beneficial for making in vitro translation a competitive technology for the production of proteins by recombinant methods. The present invention provides means of increasing the utility of cell-free protein synthesis systems by lengthening the duration of protein synthesis.

Relevant Literature

U.S. Pat. No. 6,337,191 B1, Swartz et al. Kim and Swartz (2000) Biotechnol Prog. 16:385-390; Kim and Swartz (2000) Biotechnol Lett. 22:1537-1542; Kim and Choi (2000) J Biotechnol. 84:27-32; Kim et al. (1996) Eur J Biochem. 239: 881-886. Jewett et al. (2002) Prokaryotic systems for in vitro expression, in Gene Cloning and Expression Technologies (Weiner, M. P. and Lu, Q.: eds.), Eaton Publishing, Westborough, Mass., pp. 391-411. Kim and Swartz (2000) Biotechnol Prog 16:385-390; Kim and Swartz (2000) Biotechnol Lett 22:1537-1542; Kim and Swartz (2001) Biotechnol Bioeng 74:309-316; Kim and Choi (2000) J Biotechnol 84:27-32. Davanloo, P., A. H. Rosenberg, J. J. Dunn, and F. W. Studier. 1984. Cloning and expression of the gene for bacteriophage T7 RNA polymerase. Proc Natl Acad Sci USA 81:2035-2039.

### SUMMARY OF THE INVENTION

Compositions and methods are provided for the improved in vitro synthesis of polypeptides, where the duration of detectable protein synthesis in a reaction is substantially extended over existing methods, thereby providing for increased total yield of polypeptide. UTP and CTP degradation is found to be a critical factor leading to the cessation of protein synthesis. Increased synthesis is accomplished by maintaining the concentration of CTP and UTP in the reaction mixture at a pre-determined level.

In one embodiment of the invention the reaction mixture is supplemented with additional CTP and UTP. In another embodiment of the invention, the organism from which the extracts for in vitro synthesis are obtained is genetically modified to inactivate genes encoding certain phosphatases.

In another embodiment of the invention, increased synthesis is obtained by maintaining the concentration of cysteine and serine at a pre-determined level. The reaction mixture may also be supplemented with additional amino acids during the course of the reaction.

### BRIEF DESCRIPTION OF THE DRAWINGS

FIG. 1 is a graph depicting nucleotide consumption/degradation in the cytomim system. Batch reactions synthesizing chloramphenicol acetyl transferase (CAT) were carried out for 6 hours. Nucleotide concentration profiles were measured using HPLC analysis. Error bars represent the standard deviation for 3 separate reactions. Triphosphate concentrations: Closed diamond, CTP. Open triangle, UTP. Shaded circle, GTP. Asterisks, ATP.

FIG. 2 is a graph depicting fed-batch experiments with the cytomim system. Reactions were carried out for 24 hours at 33° C. Fifteen microliter reaction mixtures were prepared in different tubes for every time point. At each time point, one tube was sacrificed in order to determine the amount of expressed protein. CAT expression was determined from ¹⁴C-leucine incorporation. Error bars represent the standard deviation from 3 to 8 separate experiments. The indicated reaction components were added at 1.5, 3.5, 6.5 and 9.5 hours. The consumed/degraded substrates were added in the following concentrations: 33 mM pyruvate, 0.5 mM CTP, 0.5 mM UTP, 1.8 mM potassium hydroxide, 0.6 mM asparagine, 0.6 mM glutamine, 0.3 mM threonine, 2.4 mM cysteine, 1.2 mM serine, 12 mM potassium glutamate, 0.05 mg/mL T7 RNA polymerase, and 0.007 mg/mL pK7CAT plasmid. The amino acid mixture contained asparagine, glutamine, threonine, cysteine, serine, and glutamate. Closed diamonds, control: no additions. Open triangles, amino acid mixture added. Open Diamonds, cysteine and serine added. Shaded squares, CTP, UTP and potassium hydroxide added. Open circles, amino acid mixture, CTP, UTP and potassium hydroxide added.

FIG. 3 is a graph depicting amino acid consumption/degradation in the cell-free protein synthesis system. Batch reactions synthesizing CAT were carried out for 6 hours using the Cytomim system. Error bars represent the standard deviation for 6-8 separate experiments. Amino acids were analyzed using an AAA-DIRECT™ system from Dionex (Sunnyvale, Calif.). All 20 amino acids were monitored over the course of the reaction and those that demonstrated the most dramatic concentration reduction are shown, with the exception of glutamate. Closed squares, glutamine. Open circles, asparagine. Asterisks, threonine. Closed triangles, serine. Closed diamonds, cysteine.

FIG. 4: 5 ml scale fed-batch experiments with the Cytomim system. Larger scale reactions were carried out at 37° C. in a 10 mL stirred glass beaker. CAT expression was determined from ¹⁴C-Ieucine incorporation. A small piece of stainless steel wire was threaded through a 30 cm long piece of silicone tubing (1.47 mm ID, 1.96 mm OD). About 15 centimeters of the tubing was immersed in the cell-free reaction mixture by coiling inside the reactor. This tubing was pressurized with pure O₂ to deliver the oxygen necessary for the regeneration of ATP within the cell-free protein synthesis reaction. The consumed/degraded substrates were added in the following concentrations: 0.5 mM CTP, 0.5 mM UTP, 1.8 mM potassium hydroxide, 0.5 mM asparagine, 0.5 mM glutamine, 2 mM cysteine, 1 mM serine, 10 mM potassium glutamate, 0.05 mg/mL T7 RNA polymerase, and 0.007 mg/mL pK7CAT plasmid. The amino acid mixture contained asparagine, glutamine, threonine, cysteine, serine, and glutamate. It was added every thirty minutes in the fed reaction. UTP, CTP, potassium hydroxide, T7 RNA Polymerase and an additional 30 mM potassium glutamate were added at 1.2, 2.7, 4.2, and 6 hours. pK7CAT was added at 1.2 and 6 hours. 33 mM pyruvate was added at 2.7 hours. The error bars represent the high and low of two separate experiments.

### DETAILED DESCRIPTION OF THE EMBODIMENTS

This invention describes the discovery of a new substrate degradation pathway that limits in vitro protein expression and a method to circumvent this obstacle for the practice of methods of in vitro protein synthesis. Although adenosine triphosphate (ATP), guanosine triphosphate (GTP), and amino acids have been previously identified as critical elements, the depletion of which leads to the termination of translation, it is shown herein that UTP and/or CTP can be rate limiting for in vitro polypeptide synthesis due to selective exhaustion from the cell free reaction. Stabilization of CTP and/or UTP concentrations during a fed-batch reaction increases yield and extends protein synthesis.

Some amino acids are also found to be depleted from the cell-free reaction. In particular, cysteine and serine were entirely degraded within the first hour of the reaction. Stabilization of the concentration of these amino also increases yield. When the stabilization of UTP and/or CTP is combined with addition of the depleted amino acids, there is a synergistic increase in the amount of protein synthesized.

In vitro synthesis, as used herein, refers to the cell-free synthesis of polypeptides in a combined transcription/translation reaction, where the reaction mix comprises biological extracts and/or defined reagents. The reaction mix will comprise a template for production of the macromolecule, e.g. DNA, mRNA, etc.; monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. Such synthetic reaction systems are well-known in the art, and have been described in the literature. A number of reaction chemistries for polypeptide synthesis can be used in the methods of the invention. For example, reaction chemistries are described in U.S. Pat. No. 6,337,191, issued Jan. 8, 2002, and U.S. Pat. No. 6,168,931, issued Jan. 2, 2001 herein incorporated by reference. Aerobic or anaerobic conditions may be used.

The present invention provides the benefit of stabilizing CTP and UTP concentrations during the synthetic reaction. In one embodiment of the invention, CTP, and/or UTP is added to the reaction mix, such that the concentration of CTP and/or UTP is stabilized at an average concentration of at least about 0.3 mM; usually at least about 0.5 mM, and may be stabilized at a concentration of 1.5 mM or higher. It will be understood by one of skill in the art that the actual concentration will fluctuate over time, as the reactants are depleted.

Methods of achieving stabilization may utilize a variety of methods. In one embodiment, the reaction is a batch process, and additional CTP and UTP are added to the reaction mixture over time, e.g. every half hour, every hour, every two hours, every four hours, etc. A reaction will usually have at least one addition of CTP and/or UTP, more usually at least two additions. CTP and UTP can also be added continuously at a slow rate.

The pH of the reaction is generally run between pH 6-9. Optionally, the pH of the reaction is maintained during addition of the nucleotides, e.g. by the addition of a base sufficient to maintain the pH at a physiological level, for example by the addition of potassium hydroxide, ammonium hydroxide, or sodium hydroxide.

An alternative method for the stabilization of CTP and/or UTP concentrations is through genetic modification of the host organism to inactivate phosphatases that degrade nucleotides. The function of phosphatases, many of which are periplasmic proteins, is to dephosphorylate a broad array of structurally diverse compounds. The presence of these phosphatases in the extract used for synthesis can result from decompartmentalization of the periplasmic and cytoplasmic spaces after cell breakage. Removing the activity of the deleterious enzymes responsible for CTP and UTP degradation is expected to improve the productivity of the system due to the lack of non-productive nucleotide degradation. Three enzymes involved in nucleotide degradation are 5′-nucleotidase (ushA), alkaline phophatase (phoA), and a nonspecific acid phophatase (aphA). In particular, 5′-nucleotidase, which has activity to hydrolyze nucleotide mono-, di-, and tri-phosphates, is a concern. Other enzymes may also be important in the CTP/UTP degradation activity present in the cell-free reaction. Genetic measures can be used to inactivate the genes that encode for the enzymes listed and others that may have deleterious activity.

The present invention also provides the benefit of stabilizing cysteine and serine concentrations during the synthetic reaction. In one embodiment of the invention, serine and/or cysteine is added to the reaction mix, such that the concentration of serine and/or cysteine is stabilized at an average concentration of at least about 0.25 mM; usually at least about 1.5 mM, and may be stabilized at a concentration of 4 mM or higher. It will be understood by one of skill in the art that the actual concentration will fluctuate over time, as the reactants are depleted.

Methods of achieving stabilization may utilize a variety of methods. In one embodiment, the reaction is a batch process, and additional serine and/or cysteine are added to the reaction mixture over time, e.g. every half hour, every hour, every two hours, every four hours, etc. A reaction will usually have at least one addition of serine and/or cysteine, more usually at least two additions. Serine and/or cysteine can also be added continuously at a slow rate.

In one embodiment of the invention, the reaction chemistry is as described in co-pending patent application US 60/404,591, filed Aug. 19, 2002, herein incorporated by reference, which may be referred to as the Cytomim system. Oxidative phosphorylation is activated, providing for increased yields and enhanced utilization of energy sources. Improved yield is obtained by a combination of factors, including the use of biological extracts derived from bacteria grown on a glucose containing medium; an absence of polyethylene glycol; and optimized magnesium concentration. This provides for a homeostatic system, in which synthesis can occur even in the absence of secondary energy sources.

The compositions and methods of this invention allow for production of proteins with any secondary energy source used to energize synthesis. These can include but are not limited to glycolytic intermediates, such as glucose, glucose-6-phosphate, fructose-6-phosphate, fructose-1,6-diphosphate, triose phosphate, 3-phosphoglycerate, 2-phosphoglycerate, phosphoenolpyruvate (PEP), and pyruvate. Any compound that can be used to generate reduction equivalents, or activate a pathway that may generate reduction equivalents may also be added. This includes amino acids, particularly glutamate, compounds in the tricarboxylic acid (TCA) cycle, citrate, cis-aconitate, isocitrate, α-ketoglutarate, succinyl-CoA, succinate, fumarate, malate, oxaloacetate, and glyoxylate, or compounds that can be directed into central metabolism (Glycolysis and the TCA cycle) Furthermore, vesicles containing respiratory chain components may also be added to assist in energy generation. It is preferable that secondary energy sources, if added, are homeostatic with respect to phosphate accumulation. The energy source may be supplied in concentrations around 30 mM. The secondary energy sources are not usually added in concentrations greater than 150 mM. Additional amounts of the energy source may be added to the reaction mixture during the course of protein expression to fuel longer reaction times. It is not necessary to add exogenous cofactors. Compounds such as nicotinamide adenine dinucleotide (NADH), NAD⁺, or acetyl-coenzyme A can be used to supplement protein synthesis yields but are not required. Addition of oxalic acid, a metabolic inhibitor of phosphoenolpyruvate synthetase (Pps), is beneficial in increasing protein yields as previously described, but is not necessary. In some cases adding oxalic acid can be harmful to the reaction. The temperature of the reaction is generally between 20° C. and 40° C. These ranges may be extended.

The combined transcription and translation system, generally utilized in E. coli systems, continuously generates mRNA from a DNA template, which can be in the form of a plasmid or PCR fragments, with a recognizable promoter. Either endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Nucleases can be removed from extracts to help stabilize mRNA levels. The template can encode for any particular gene of interest.

The reactor configuration for synthesis is not limited to the batch configuration. The realization that UTP and CTP can energize protein synthesis would be useful in designing optimal feeding solutions for continuous exchange or semi-continuous systems.

Metabolic inhibitors for undesirable enzymatic activity may be added to the reaction mixture. Alternatively, enzymes or factors that are responsible for undesirable activity may be removed directly from the extract or the gene encoding the undesirable enzyme may be inactivated or deleted from the chromosome.

The particular strain of bacteria utilized for the development of this new technology may be changed. In particular, genetic modifications can be made to the strain that can enhance protein synthesis. For example, the strain utilized in the experiments described above had the speA, tnaA, tonA, and endA genes deleted from the chromosome. Respectively, these mutations helped to stabilize arginine concentration, stabilize tryptophan concentration, protect against bacteriophage infection, and stabilize DNA within the system.

Other salts, particularly those that are biologically relevant, such as manganese, may also be added. Potassium is generally added between 50-250 mM, ammonium between 0-100 mM, and magnesium between 6-15 mM. The pH of the reaction is generally run between pH 6-9. The temperature of the reaction is generally between 20° C. and 40° C. These ranges may be extended.

Vesicles, either purified from the host organism or synthetic, may also be added to the system. These may be used to enhance protein synthesis and folding. The Cytomim technology has been shown to activate processes that utilize membrane vesicles. Inverted vesicles containing respiratory chain components must be present for the activation of oxidative phosphorylation and, in the Cytomim system, are present in an active form from the S30 cell extract. The present methods may be used for cell-free expression to activate other sets of membrane proteins.

### Reaction Chemistry

Synthetic systems of interest include the replication of DNA, which may include amplification of the DNA, the transcription of RNA from DNA or RNA templates, the translation of RNA into polypeptides, and the synthesis of complex carbohydrates from simple sugars.

The reactions may be large scale, small scale, or may be multiplexed to perform a plurality of simultaneous syntheses. Additional reagents may be introduced to prolong the period of time for active synthesis. Synthesized product is usually accumulated in the reactor, and then is isolated and purified according to the usual method for protein purification after completion of the system operation.

Of particular interest is the translation of mRNA to produce proteins, which translation may be coupled to in vitro synthesis of mRNA from a DNA template. Such a cell-free system will contain all factors required for the translation of mRNA, for example ribosomes, amino acids, tRNAs, aminoacyl synthetases, elongation factors and initiation factors. Cell-free systems known in the art include E. coli extracts, etc., which can be treated with a suitable nuclease to eliminate active endogenous mRNA.

In addition to the above components such as cell-free extract, genetic template, and amino acids, materials specifically required for protein synthesis may be added to the reaction. These materials include salts, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, etc.

The salts preferably include potassium, magnesium, ammonium and manganese salt, acetic acid or sulfuric acid, and some of these may have amino acids as a counter anion. The polymeric compounds may be polyethylene glycol, dextran, diethyl aminoethyl dextran, quaternary aminoethyl and aminoethyl dextran. The oxidation/reduction adjuster may be dithiothreitol, ascorbic acid, glutathione and/or their oxides. Also, a non-denaturing surfactant such as Triton X-100 may be used at a concentration of 0-0.5 M. Spermine and spermidine may be used for improving protein synthetic ability, and cAMP may be used as a gene expression regulator.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

Preferably, the reaction is maintained in the range of pH 5-10 and a temperature of 20°-50° C., and more preferably, in the range of pH 6-9 and a temperature of 25°-40° C.

The amount of protein produced in a translation reaction can be measured in various fashions. One method relies on the availability of an assay which measures the activity of the particular protein being translated. Examples of assays for measuring protein activity are a luciferase assay system and a chloramphenical acetyl transferase assay system. These assays measure the amount of functionally active protein produced from the translation reaction. Activity assays will not measure full length protein that is inactive due to improper protein folding or lack of other post translational modifications necessary for protein activity.

Another method of measuring the amount of protein produced in coupled in vitro transcription and translation reactions is to perform the reactions using a known quantity of radiolabeled amino acid such as ³⁵S-methionine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Experimental

### EXAMPLE 1

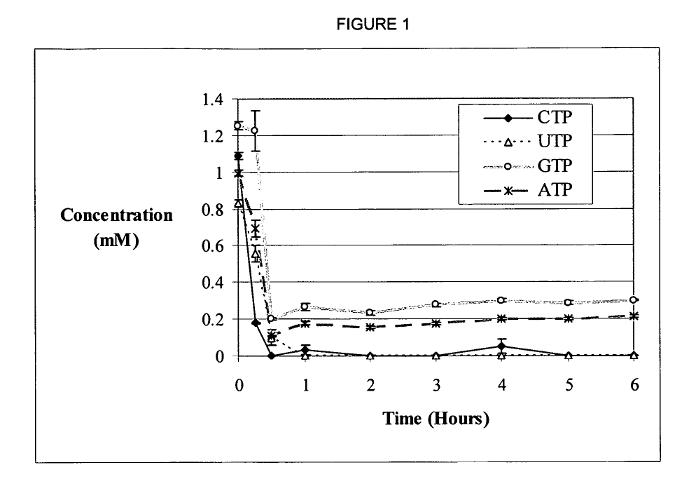

A new substrate degradation pathway that limits in vitro protein expression is discovered, along with methods to circumvent this obstacle. Adenosine triphosphate (ATP), guanosine triphosphate (GTP), and amino acids have been previously identified as critical elements, the depletion of which leads to the termination of translation. In the case of the Cytomim system, ATP and GTP concentrations are not limiting as they remained relatively steady over the course of a protein biosynthesis reaction (shown in FIG. 1).

Strikingly, although concentrations of ATP and GTP were constant, uridine and cytidine triphosphate (UTP and CTP, respectively) were entirely depleted during the first hour of protein synthesis in vitro (shown in FIG. 1). This is the first evidence of such nucleotide degradation in cell-free protein synthesis systems. This result is especially surprising since the two other nucleotides, ATP and GTP, are present at greater than 200 μM for the majority of the reaction.

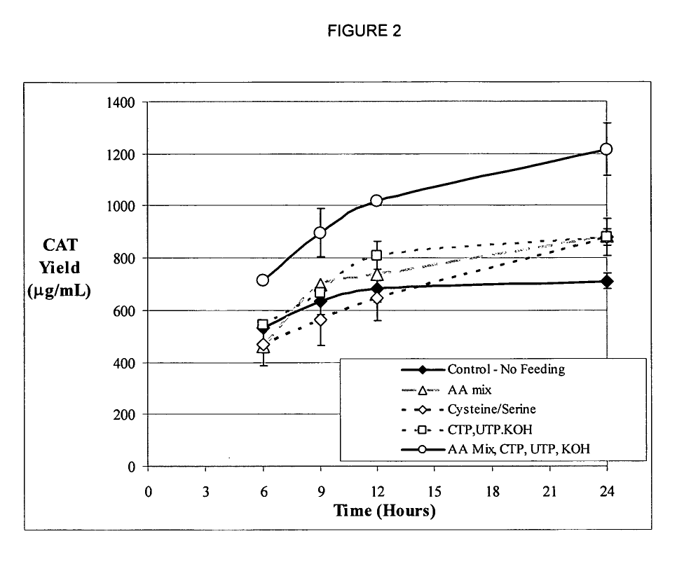

The data indicate that CTP and UTP are selectively exhausted from the cell free reaction. Repeated additions of CTP and UTP during a fed-batch reaction increase protein synthesis yields by 25% and extend protein synthesis as compared to the un-fed Cytomim system (shown in FIG. 2, and as described in the figure legends). From this it can be concluded that degradation of these two small molecule substrates limits the protein production duration for the cell-free protein synthesis system. Because the addition of UTP and CTP alone led to a decrease in the pH of the system, potassium hydroxide was also added in order to maintain a homeostatic pH.

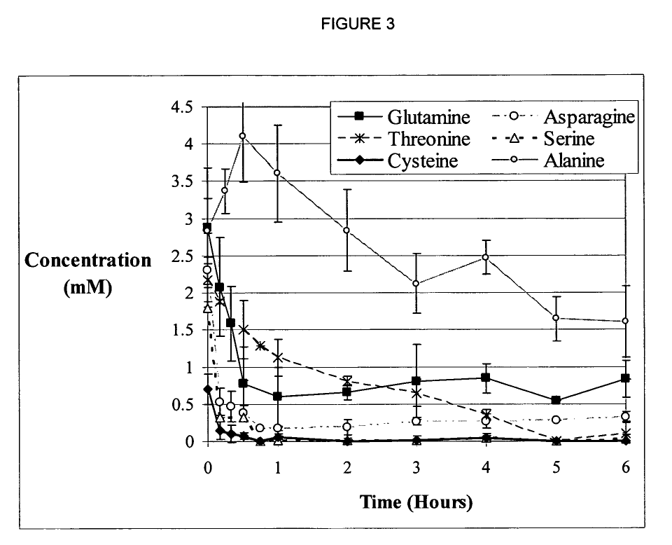

Some amino acids were also depleted from the cell-free reaction (shown in FIG. 3). In particular, cysteine and serine were entirely degraded within the first hour of the reaction. When the feeding of UTP, CTP, and potassium hydroxide is combined with the addition of consumed amino acids in a fed-batch reaction, protein synthesis yields were significantly enhanced by approximately 75% over a 24 hour reaction, to approximately 1.2 mg CAT/ml (shown in FIG. 2). This amount of protein produced is greater than the current benchmark (1 mg/mL) for creating a process that is commercially viable.

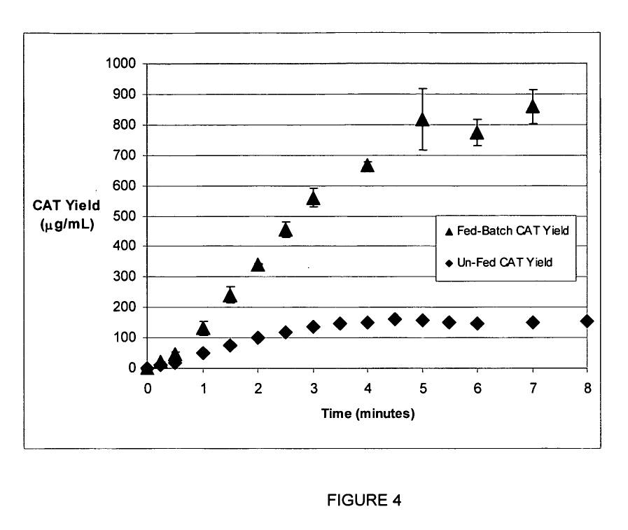

It is interesting to note that feeding amino acids alone increased yields approximately the same amount as the additions of CTP and UTP, by about 25%. However, when CTP, UTP, and amino acid feeding were combined, there was a synergistic increase in the amount of protein synthesized. Adding cysteine and serine provided the same benefit for synthesis as adding the entire amino acid mixture (shown in FIG. 2). Applying this methodology to a larger scale cell-free reaction, 5 ml, also improved yields (shown in FIG. 4). These data demonstrate a significant advantage to be gained by incorporating UTP and CTP feeding into cell-free reaction protocols. Moreover, this large scale reaction produced about 150 nanomoles of CAT, which is enough to be used for NMR structure determination.

Materials and Methods

The standard reaction mixture for a coupled transcription-translation reaction used in these experiments contains the following components: 1.2 mM ATP, 0.85 mM each of GTP, UTP and CTP, 130 mM potassium glutamate, 10 mM ammonium glutamate, 8 mM magnesium glutamate, 1.5 mM spermidine, 1 mM putrescine, 34 μg/ml folinic acid, 170.6 μg/ml E. coli tRNA mixture, 13.3 μg/ml plasmid, 100 μg/ml T7 RNA polymerase, 2 mM each of 20 unlabeled amino acids, 5 μM [¹⁴C leucine, 0.33 mM nicotinamide adenine dinucleotide, 0.26 mM Coenzyme A, 2.7 mM sodium oxalate and 0.24 volumes of S30 extract.

Prokaryotic cell-free protein synthesis is performed using a crude S30 extract derived from Escherichia coli K12 (strain A19 ΔtonA ΔtnaA ΔspeA ΔendA met+), with slight modifications from the protocol of Pratt (Jewett et al., in Gene Cloning and Expression Technologies, 2002). Specifically, the extract is grown with 2×YTPG media (Kim and Choi) containing glucose and phosphate as compared to the more standard 2×YT medium. T7 RNA polymerase was prepared from E. coli strain BL21 (pAR1219) according to the procedures of Davanloo et al., 1984. Plasmid pK7CAT was used as a template for protein synthesis. pK7CAT encodes for the sequence of chloramphenicol acetyl transferase (CAT) using the T7 promoter and terminator.

Batch reactions (FIG. 1) were incubated at 37° C. for 6 hours. Fed-batch reactions (FIG. 2) were incubated for 24 hours at 33° C. Fifteen microliter reaction mixtures were prepared in different tubes for every time point. At each time point, one tube was sacrificed in order determine the amount of expressed protein or nucleotide and/or amino acid concentration. The amount of synthesized protein is estimated from the measured TCA-insoluble radioactivities using a liquid scintillation counter (Beckman LS3801).

The consumed/degraded substrates were added in the following concentrations during the fed-batch reactions: 0.5 mM CTP, 0.5 mM UTP, 1.8 mM potassium hydroxide, 0.6 mM asparagine, 0.6 mM glutamine, 0.3 mM threonine, 2.4 mM cysteine, 1.2 mM serine, 12 mM potassium glutamate, 0.05 mg/mL T7 RNA polymerase, and 0.007 mg/mL pK7CAT plasmid. The amino acid mixture contained asparagine, glutamine, threonine, cysteine, serine, and glutamate. Adding only cysteine and serine provided the same benefit for synthesis as adding the entire amino acid mixture (FIG. 2). Reaction components were added at 1.5, 3.5, 6.5 and 9.5 hours.

High performance liquid chromatography (HPLC) analysis was used for the nucleotide degradation data. For the analysis, a five percent TCA solution was added to the cell extract reaction mixture in a 1:1 volumetric ratio. TCA precipitated samples were centrifuged at 12,000 g for 10 minutes at 4° C. The supernatant was collected. Twenty microliter samples were applied to a Vydac column for HPLC analysis. The nucleotide column 302IC4.6 (Vydac, Hesperia, Calif.) and an Agilent 1100 series HPLC system were used. (Palo Alto, Calif.) Separation was carried out at a flow rate of 2 ml/min. The mobile phase started with 100% of a 25 mM phosphate buffer (1:1 molar ratio of NaH₂PO4/Na₂HPO₄ adjusted to pH 2.6 with glacial acetic acid) and 0% of a 125 mM phosphate buffer solution (1:1 molar ratio of NaH₂PO₄/Na₂HPO₄ adjusted to pH 2.8 with glacial acetic acid). A linear gradient of 0% to 100% of the 125 mM phosphate buffer was applied from 2 to 25 minutes, then maintained at 100% for 2 minutes and returned to 0% in a linear gradient over three minutes. Nucleotides were detected at 260 nm. Nucleotide concentrations were determined by comparison to a calibration obtained with nucleotide standards.

Amino acids were analyzed using an AAA-DIRECT™ system from Dionex. (Sunnyvale, Calif.) Five percent TCA was added to the cell extract reaction mixture in a 1:1 volumetric ratio. TCA precipitated samples were centrifuged at 12,000 g for 10 minutes at 4° C. The supernatant was collected and diluted 250 times. Twenty microliter samples were applied to an AMINOPAC™ column for HPLC analysis. The specifically designed method used gradient anion exchange for component separation. Amino acids were detected using a gold working electrode with Pulsed Electrochemical Detection (PED). Amino acid concentrations were determined by comparison with a calibration standard.

1. A method for enhanced synthesis of polypeptide in vitro, the method comprising:
synthesizing said polypeptides in a coupled transcription/translation in a reaction mix where the concentration of UTP and/or CTP is maintained at a pre-determined average concentration.

2. The method according to claim 1, wherein said pre-determined average concentration is at least about 0.25 mM.

3. The method according to claim 1, wherein the concentration of both CTP and UTP is maintained.

4. The method according to claim 3, further comprising the step of maintaining the pH of the reaction.

5. The method according to claim 4, further comprising the step of maintaining the concentration of serine and/or cysteine at a pre-determined average concentration.

6. The method according to claim 5, wherein said step of maintaining the concentration of serine and/or cysteine provides for a synergistic increase in protein synthesis.

7. The method according to claim 6, wherein said reaction activates oxidative phosphorylation.
